(19) United States
(12) Patent Application Publication
(10) Pub. No.: US 2002/0082575 A1
(43) Pub. Date: Jun. 27, 2002

(54) Absorbent articles

(76) Inventors: Ervin Dan, Calgary (CA); Arthur Roger Shipley, Clwyd (GB)

Correspondence Address: Martin G. Linihan, Hodgson Russ LLP, Suite 2000, One M&T Plaza, Buffalo, NY, 14203-2391, US

(21) Appl. No.: 10/072,023

(22) Filed: Feb. 7, 2002

Related U.S. Application Data
(63) Continuation of application No. 09/011,539, filed on May 6, 1999.
Application No. 09/011,539 is a 371 of international application No. PCT/EP96/03530, filed on Aug. 9, 1996.

(30) Foreign Application Priority Data
Aug. 11, 1995 (GB) 9516447.1
Dec. 1, 1995 (GB) 9524570.0
Jul. 12, 1996 (GB) 9614668.3

(51) Int. Cl. A61F 13/15; A61F 13/20

(52) U.S. Cl. 604/378; 604/383

(57) ABSTRACT

A multiple layer water-absorbent core is described which is suitable for use in an absorbent article. The water-absorbent core comprises a water-absorbent layer and a water-management layer which acts to reduce the exposure of the core to the water. The water-management layer is preferably formed from the hydrophobic material. An absorbent article comprising a liquid pervious cover sheet, a liquid impervious backsheet and a multiple layer water-absorbent core of the invention is also described.

The present invention relates to absorbent articles. In particular, it relates to articles such as sanitary napkins and pads, incontinence garments and disposable diapers. In addition, the present invention relates to a water-management layer for an absorbent article.

As used herein, the term “water” when used alone or in the phrases “water-absorbing”, “water-absorbent”, “water-swellable” and “water-management” is understood to mean not only water but also aqueous media such as, in particular, electrolyte solutions such as body fluids.

Sanitary napkins, pads, incontinence garments and disposable diapers have been known for many years and much effort has been made to improve the functional efficiency of such articles to make them more absorbent, more comfortable to wear and less obtrusive to the wearer.

In general, such products have a core which includes a water-absorbent layer. This water-absorbent layer may be formed from any suitable water-absorbent material including wood pulp, rayon and tissue. Additionally, or alternatively, the layer may comprise any of the water-absorbing polymer compositions commonly known as superabsorbent polymers.

A number of absorbent compositions have been developed which exhibit the capacity to be water-absorbing. Known compositions may be in any suitable form including powders, particles and fibers. U.S. Pat. No. 3,954,721 and 3,983,095, which are incorporated herein by reference, disclose preparations for derivatives of copolymers of maleic anhydride with at least one vinyl monomer in fibrous form. The fibrous copolymers are rendered hydrophillic and water-swellable by reaction with ammonia or an alkali metal hydroxide. U.S. Pat. No. 3,810,468, which is incorporated herein by reference, discloses lightly cross-linked olefin-maleic anhydride copolymers prepared as substantially linear copolymers and then reacted with a diol or a diamine to introduce cross-linking. The resultant lightly cross-linked copolymers are treated with ammonia or an aqueous or alcohol solution of an alkali metal hydroxide. U.S. Pat. No. 3,980,663, which is incorporated herein by reference, describes water-swellable absorbent articles made from carboxylic polyelectrolytes via cross-linking with glycerine diglycidyl ether.

European Published Application No. 0 268 498 (incorporated herein by reference) describes a water-absorbent composition formed by causing a substantially linear polymer of water-soluble ethylenically unsaturated monomer blends comprising carboxylic and hydroxylic monomers to cross-link internally.

Further examples of water-absorbent compositions are those produced from a copolymer of an α,β unsaturated monomer having at least one pendant unit selected from a carboxylic acid group and derivatives thereof and a copolymerisable monomer. A proportion of the pendant units are present in the final copolymer as the free acid and a proportion as the salt of the acid. These copolymers are capable of being cross-linked, either internally or with a variety of cross-linking agents, to form the water-swellable composition. Examples of water-swellable compositions of this type can be found in U.S. Pat. Nos. 4,616,063, 4,705,773, 4,731,067, 4,743,244, 4,788,237, 4,813,945, 4,880,868 and 4,892,533 and EP 0 272 074 0 264,208 and 0,436,514 which are incorporated herein by reference.

Derivatives of carboxylic acid groups include carboxylic acid salt groups, carboxylic acid amide groups, carboxylic acid imide groups, carboxylic acid anhydride groups and carboxylic acid ester groups.

Other examples of water-absorbent compositions can be found in U.S. Pat. No. 4,798,861, WO 93/17066, WO 93/255735, WO 93/24684, WO 93/12275, European Published Application Nos. 0 401 044, 0 269 393, 0 326 382, 0 227 305, 0 101 253, 0 213 799, 0 232 121, 0 342 919, 0 233 014, 0 268 498 and 0 397 410, British Patent Application Nos. 2 082 614, 2 022 505, 2 270 030, 2 269 602 and 2 126 591, U.S. Pat. Nos. 4,418,163, 4,418,163, 3,989,586, 4,332,917, 4,338,417, 4,420,588 and 4,155,957 and French Patent Application No. 2 525 121 which are all incorporated herein by reference.

Water-absorbent material of the kinds referred to in the above-mentioned patents and applications may be in any suitable form including powder, particulate granular and fibers; the fibers may be straight or may be curled and/or crimped. Details of such curly/crimped fibers may be found in U.S. Pat. Nos. 4,822,453, 4,888,453 5,462,793 and 4,898,462 which are incorporated herein by reference. In one alternative material, the water-absorbent polymer may be coated onto the whole or a part of the surface of other materials such as non-water-absorbent fibers. Details of one type of fibers of this type may be found in WO 96/15307 which is incorporated herein by reference. Other kinds of water-absorbent materials may be used as, or as part of, the water-absorbing layer. Suitable materials include naturally occurring water-absorbent materials. One such water-absorbent material is starchy material such as that proposed by the US Department of Agriculture in 1969-1970. Peat moss may also be used a water-absorbent material. In this connection, reference may be made to U.S. Pat. Nos. 5,477,627 5,429,242 5,374,260 4,992,324 4,676,871 4,573,988 4,560,372 4,540,454 4,537,590 and 4,226,237 which are incorporated herein by reference. Alginates have also been proposed as suitable water-absorbent material. One example of the use of such alginates is that suggested by Beghin/Kayserberg.

The water-absorbent layer may be of non-uniform configuration. For example, where the absorbent article is a feminine hygiene product or a diaper, the water-absorbent layer may be shaped such that it is thicker in the crotch region.

Whatever material is used for the absorbent layer, it is generally backed by a fluid-impervious backing sheet to protect clothing and the surrounding area from soiling and to prevent leakage of the body fluid which would cause embarrassment to the user. Any water-impervious backing material may be used.

The article generally has a water-permeable non-woven type cover-sheet which defines the surface of the article which will, in use, be in contact with the user. The cover sheet is intended to insulate the wearer from contact with water that has been absorbed into the core, thus the cover sheet should allow water to pass through it into the core but remain dry and soft to the touch. Any suitable material may be used as the cover-sheet. One example of a suitable cover-sheet is described in U.S. Pat. No. 5,257,982 which is incorporated herein by reference.

As there is often a discharge of a substantial volume of water in a short time, the core may additionally include a distribution layer which is generally located above the water-absorbent layer. This distribution layer serves to wick the water away from the point of initial contact with the article and transport it to other parts of the water-absorbent layer. The use of a distribution layer of this type is advantageous in addressing the problem of pooling if a substantial volume of water is discharged in a short time and thereby gives the user a feeling of dryness. Examples of distribution layers include those described in EP 0 565 606, GB 2 266 465 and GB 2 278 371 which are incorporated herein by reference.

As the distribution layer serves to wick the water away from the point of initial contact, it is accepted that the water should be transported in the plane of the layer as quickly as possible and thus, distribution layers are generally made of hydrophillic material. The concept of speeding up the rate at which water is transported in the plane of the distribution layer is discussed in detail in EP 0 565 606 assigned to The Procter & Gamble Company in which the distribution layer forms part of the multiple layer absorbent core.

Whilst the use of distribution layers of this type go some way to reducing the effects of pooling, they do not satisfactorily overcome the problems of rewet once the water has been absorbed into the water-absorbent layer. Rewet is a measure of how dry the articles feel to the touch after water-absorption. This is generally measured under load of approximately 3-5 kPa. Rewet is of particular importance in the applications to which the absorbent articles of the present invention are directed as if the article feels damp, the user will feel damp and uncomfortable.

The problem of rewet is particularly acute where the water-absorbent layer is formed from or includes the water-absorbent polymers described above, in particular where it is formed from fast absorbing water-absorbent polymers. Although the water-absorbent polymers described above can absorb a large volume of water and absorption can take place in a short time period, in some circumstances, the water-absorbent polymeric may become locally saturated because of the fast absorbency of the water-absorbent and may thereby feel damp to the touch.

We have now discovered that an alternative distribution layer may be provided which satisfactorily transports the water away from the initial point of contact and overcomes the aforementioned problems of rewet if the distribution layer is formed from material which slows down the rate at which the water is transported through the layer with respect to the rate at which the water is transported in the plane of the layer. The distribution layer will hereinafter be referred to as a water-management layer to distinguish it from conventional distribution layers.

An hydrophobic layer has been found to be a particularly effective alone or as a component of a water-management layer. This discovery is particularly surprising since it would be expected that an hydrophobic water-management layer would reduce the ability of the layer to remove water from the point of contact.

Thus, according to one aspect of the present invention there is provided a water-management layer which is suitable for use in conjunction with a water-absorbent layer in an absorbent article wherein the water-management layer acts to reduce the exposure of the water-absorbent layer to water. That is to say that the rate of transport of the water through the layer is slower than in conventional distribution layers. This allows the water to spread through/along the layer before it passes through the layer.

By “exposure” we mean the time that elapses between the water being applied to the surface of the water-management layer and its entering the water-absorbent layer.

The material from which the water-management layer is preferably formed is a hydrophobic material or a material made hydrophobic by being treated with a suitable agent, for example a silicone.

Where the water-absorbent layer is used in conjunction with a water-management layer, a multi-layered core is obtained. Thus, according to a second aspect of the present invention there is provided a multi-layered core suitable for use in an absorbent article comprising the water-management layer of the above first aspect of the present invention and a water-absorbent layer wherein the water-management layer is located above the water-absorbent layer.

The water-absorbent layer of the core preferably includes a water-absorbent polymeric composition which may be in the form of fibers.

Any material which allows the water to spread along the plane of the layer before it passes through the layer may be used. One suitable material from which the water-management layer may be formed is an apertured film. Examples of apertured films include those described in WO 94/28846, U.S. Pat. No. 5,478,335, DE 959814, EP 0 598 970, U.S. Pat. Nos. 4,806,303, 4,626,254, 4,634,440, 5,180,620, 5,387,209, 5,449,352, IT 591405, U.S. Pat. Nos. 4,895,749, 5,078,710 and EP 0 195 113 and those described by Smith & Nephew, AET (under the trade name HERCULES), Johnson & Johnson (under the trade name RETICULON), Pollguf, Evial, Plastik, EPG, Avgul and Honshu. Each of the patents and applications detailed are incorporated herein by reference as are the commercial products mentioned.

A preferred apertured film is a plastics, preferably polypropylene, film having spaced holes. The polypropylene film may have from about 50 holes per square inch to about 250 holes per square inch, preferably 100 to 180, more preferably 130 to 150 holes per square inch. 140 holes per square inch may also be used.

In a particularly preferred embodiment, some or all of the holes have a three-dimensional structure, most preferably, some or all of the holes are fluted, ie funnel shaped. Fluted holes may be formed by any appropriate means. One suitable means is a method is which the polymeric sheet is placed over a grid having spaced holes and heating the sheet whilst applying a vacuum to the side of the grid remote from that of the polymeric sheet. As the sheet is heated it softens and material is pulled, by the force of the vacuum, through the holes until the flutes formed burst at their bases thereby forming the apertured film. One alternative means involves treating the polypropylene film with hot needles. The fluted holes are preferably configured such that the base of the apertures extend beneath the bottom surface of the plane of the film. Thus, when the apertured film is used in the absorbent article of the present invention the bottom of the fluted apertures preferably penetrate the water-absorbent layer. However, the flutes may also be oriented such that they are directed away from the water-absorbent layer in use.

In one alternative arrangement, the apertured film may comprise a plurality of sheets each having holes. The holes of each sheet may be of different diameters and configured such that when the sheets are laid one on another, the holes “line-up” to form an aperture of non-cylindrical, preferably fluted, cross section.

The apertured polymeric film may be corrugated.

One alternative suitable material for the water-management layer is a non-woven material that is made hydrophobic and thereby slows down the rate of transport of water through the layer which thereby forces lateral water-management of the water.

The non-woven material may be any suitable non-woven material that has been treated such that it becomes hydrophobic. Any means of making the non-woven material hydrophobic may be used. One example of a suitable treatment is spraying the material with a silicone composition. The non-woven material may be made by any suitable means from non-water-absorbent fibers. Suitable means include melt-blown and spun-bonded methods. By “non-water-absorbent” we mean that the fibers do not absorb to an appreciable extent. Suitable materials from which the non-woven material may be formed include natural or synthetic fibers such as cellulose, viscose, polyester, non-water-absorbent polymers of propylene, polyamide and ethylenepropylene copolymer fibers, and mixtures thereof and the like, with polyester, polyethylene and polypropylene fibers being particularly preferred.

Additionally, or alternatively, the water-management layer, howsoever formed, may be or include arrangements sometimes known as “lateral movers” located on the upper surface of the layer. Suitable lateral movers include creped tissue, embossed tissue (additionally or alternatively, the water-management layer itself may be embossed), a “Burgeni” skin arrangement for example that described in U.S. Pat. No. 2,925,260, under layers such as those described in EP 0 523 719, U.S. Pat. No. 5,437,653, WO 92/09716 and U.S. Pat. No. 5,415,640, the arrangement described in U.S. Pat. No. 2,945,386 and the arrangements in WO 93/11725, U.S. Pat. Nos. 5,411,497, 5,425,725 and 5,433,715. Each of the above-mentioned patents and applications are incorporated herein by reference.

In a particularly preferred embodiment fibers are located on the upper surface of the water-management layer. The fibers may be individual fibers or a non-woven or woven mat of fibers. The fibers in the mat may be bonded. Without wishing to be bound by any particular theory it is believed that these fibers create a lofted area that behaves as capillaries for the lateral movement of the fluid. The fibers may behave as capillaries for this lateral movement. The fibers may be arranged such that they generally extend away from the water-management layer such that when in use in an absorbent article, the fibers are directed towards the cover sheet where present. The fibers may be arranged such that there are voids between the fibers. The fibers may, in one embodiment, be bonded to each other.

In a preferred arrangement there are grooves or channels formed between the fibers or between groups of fibers. The arrangement of the fibers such that there are grooves or channels present may be achieved by any suitable means. However, carding is particularly preferred. The grooves or channels preferably run substantially parallel to the longitudinal edge of the -absorbent article. For example, where the absorbent article is a sanitary towel or pant liner, the grooves or channels will run lengthways along the pad.

In one alternative arrangement the fibers themselves may be grooved. In this connection, reference may be made to Eastman Chemicals 4DG fibers.

The fibers may be any suitable material and are preferably selected from rayon fibers, cellulose ester fibers, protein fibers, polyamide fibers, polyester fibers, polyvinyl fibers, polyolefin fibers, polyurethane fibers, aramid fibers, glass fibers and mixtures thereof. Particularly useful fibers are fibers having a hollow core such as the polyester, typically polyester terephthalate, fibers commercially available from E. I. DuPont de Nemours under the trade name HOLLOWFILL. Bicomponent fibers may also be used.

The fibers may be of any suitable size. However, the fibers are preferably from about 15 to about 50 mm in length, or longer, more preferably 20 to 35 mm in length, still more preferably 25mm in length.

According to a third aspect of the present invention, there is provided an absorbent article including the multi-layered core of the above second aspect.

According to a further aspect of the present invention, there is provided an absorbent article comprising a liquid pervious cover sheet, a liquid impervious backsheet and a multi-layered core described in the above second aspect wherein the cover sheet is joined to the backsheet to enclose the core.

The layers of the absorbent article are preferably bonded to adjacent layers. Thus, for example, the water-absorbent layer will be bonded to the water-management layer and the water-management layer will be bonded to the cover sheet. Where the water-management layer additionally or alternatively includes fibers located on its upper surface, the fibers are preferably bonded to the water-management layer. Further, when the absorbent article has a cover sheet, the fibers are preferably bonded to the cover sheet using a sprayable adhesive which is preferably a hot-melt adhesive. Thus, the fibers may be bonded to the water-management layer, to the cover sheet, or to the water-management layer and the cover sheet. Where individual fibers are used, each fiber may be bonded individually. Where a mat of fibers is used, it may only be necessary to bond portions of the mat to the adjacent layers.

The adhesive may be applied such that it forms a pattern, wherein the spaces between the nodules of adhesive may form grooves and channels which may serve to direct the fluid laterally. Thus, an adhesive may be used as one alternative to, or in addition to, the fibers described above.

In addition to reducing the pooling effects discussed above, the water-management layer of the present invention may also act to reduce rewet. Without wishing to be bound by any theory, it is believed that the water-management layer of the present invention allows water to pass through it in a controlled manner as discussed above and then behaves as an enclosure to prevent water in the water-absorbent layer from coming into contact with the user.

A particularly preferred embodiment of the present invention will now be described, with reference to the accompanying drawings in which:

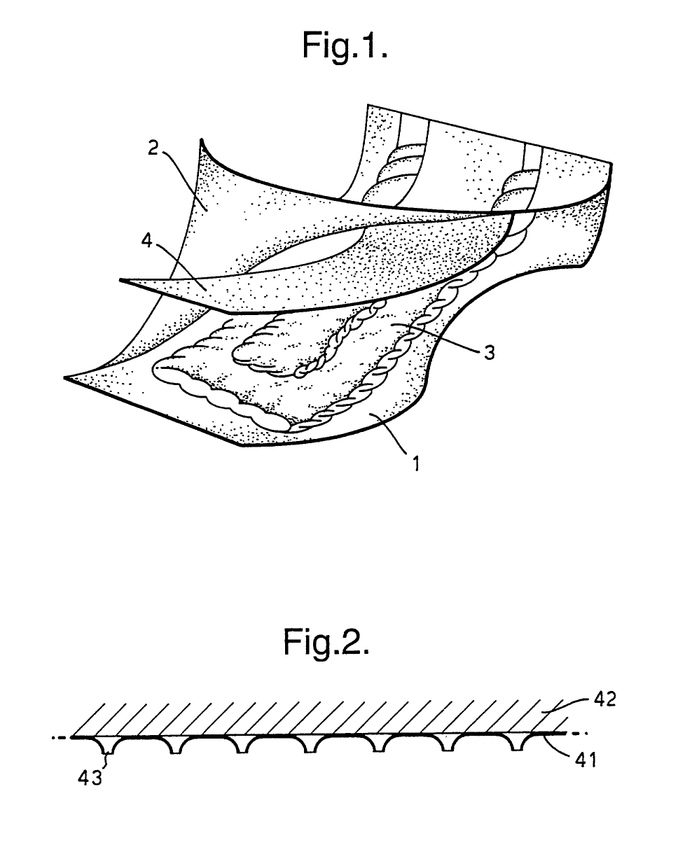

FIG. 1 is a schematic representation of a diaper including the water-management layer of the present invention; and

FIG. 2 is a schematic representation of one water-management layer of the present invention.

The diaper as illustrated in FIG. 1 is of simple construction for ease of understanding. It will be understood that the diaper may be of any configuration and may include features to aid in the fitting of the diaper to the baby, including elastication and closure tabs. The diaper comprises a polyethylene backsheet 1 and a non-woven cover stock 2 which are joined around their peripheries in conventional manner to enclose a three dimensional water-absorbent layer 3. The water-absorbent layer may be of any suitable arrangement and may consist of, for example, bulking material such as wood fluff and water-absorbent material.

The water-absorbent material is preferably water-absorbent fibers formed from a syrup comprising an a,B unsaturated monomer having at least one pendant unit selected from a carboxylic acid group and derivatives thereof and a copolymerisable monomer. A proportion of the pendant units are preferably present in the final copolymer as the free acid and a proportion as the salt of the acid. These copolymers are capable of being cross-linked, either internally or with a variety of cross-linking agents, to form the water-absorbent composition.

Preferably before being placed in the diaper, the core may be compressed and then sprayed with a quantity of a binder, an adhesive and/or water.

Located above the water-absorbent layer is a water-management layer 4 in accordance with the first aspect of the present invention. The water-management layer 4 can be further described with reference to FIG. 2. The water-management layer of FIG. 2 comprises a polypropylene apertured film 41 having 130 holes per square inch. The holes are formed by the hot-needle method and therefore are of fluted cross-section. Fibers 42 are located on the surface of the apertured film. One end of each fiber is preferably bonded to the apertured film. These fibers form a high loft area.

When the water-management layer is placed on the water-absorbent layer, the base of each fluted hole preferably penetrates into the top of the water-absorbent layer. The water-management layer is preferably bonded to the water-absorbent layer.

The present invention will now be described with reference to the accompanying examples.

In the following examples, the rewet was measured by weighing 10 filter papers, placing these in contact with the cover-sheet for 15 secs under a pressure of 0.5 psi. The papers were then reweighed and the difference calculated between the weight of the papers before and after application to the coversheet. The result is a measure of the rewet.

### EXAMPLE 1

A two layer core was formed from an 20 g absorbent layer comprising 5 g of superabsorbent fibers and, located above the core, a water-management layer comprising a 20 micrometer thick polypropylene sheet having 130 holes per square inch and a 50 gsm air through carded polyester fibrous layer. The layers were bonded together. The cover-sheet from a commercial diaper was placed onto the water-management layer. 70 mls of synthetic urine were applied to the coversheet from a burette at a rate of 1 ml/s. After 20 minutes the process was repeated. After a further 20 minutes the process was again repeated. After a further 20 minutes the rewet was calculated and found to be 0.3 g.

### EXAMPLE 2

A two layer core was formed from an 20 g absorbent layer comprising 5 g of superabsorbent fibers and, located above the core, a water-management layer comprising a high loft distribution layer sprayed with a silicone. The water-management layer was bonded to the absorbent core. The cover-sheet from a commercial diaper was placed onto the water-management layer. 70 mls of synthetic urine were applied to the coversheet from a burette at a rate of 1 ml/s. After 20 minutes the process was repeated. After a further 20 minutes the process was again repeated. After a further 20 minutes the rewet was calculated and found to be 1 g.

### EXAMPLE 3

A two layer core was formed from an 20 g absorbent layer comprising 5 g of superabsorbent fibers and, located above the core, a water-management layer comprising a hydrophobic spun bond layer of approximately 30 gsm having a 50 gsm air through carded polyester fibrous layer located above. The water-management layer was bonded to the absorbent core. The cover-sheet from a commercial diaper was placed onto the water-management layer. 70 mls of synthetic urine were applied to the coversheet from a burette at a rate of 1 ml/s. After 20 minutes the process was repeated. After a further 20 minutes the process was again repeated. After a further 20 minutes the rewet was calculated and found to be 1g.

### Comparative Example 1

An absorbent core of the type used in Examples 1 and 2 was covered with the cover-sheet from a commercial diaper. 70 mls of synthetic urine were applied to the coversheet from a burette at a rate of 1 ml/s. After 20 minutes the process was repeated. After a further 20 minutes the process was again repeated. After a further 20 minutes the rewet was calculated and found to be between 10 and 20 g.

Whilst the present invention has been described with reference to sanitary napkins and pads, incontinence garments and disposable diapers it will be understood that the invention is equally applicable to other products which require high water-absorption capability such as pant liners, training pads, tampons, adult incontinence pads, bandages, patient underpads (for example pads of the type described in U.S. Pat. Nos. 3,814,101 4,342,314 and EP 0 052 403 which are incorporated herein by reference), mortuary pads, casket liners, forensic examination pads, meat trays, soaker pads for food use, medical tray pads, fenestration drapes, other medical related articles, seed germination pads, capillary mats, baby bibs, desiccant strips for anti-rust use, bath mats, packaging, sorbents, clothing, breast pads, underarm pads, surgical and dental sponges, bandages, industrial wipes, domestic wipes, wipes, filters, cable wrap, food preservation articles, roofing materials, automotive trim, furniture, gasket, sealants, pond liners, bedding, clothing, cement, household pet litter, soil modifiers, wound covers and the like.

1. A multiple layer water-absorbent core suitable for use in an absorbent article comprising a water-absorbent layer and a water-management layer which acts to reduce the exposure of the core to water wherein the water-management layer is located above the water-absorbent layer.

2. A multiple layer water-absorbent core according to claim 1 wherein the water-management layer is formed from a hydrophobic material.

3. A multiple layer water-absorbent core according to claim 1 or 2 wherein the water-management layer is an apertured film.

4. A multiple layer water-absorbent core according to claim 3 wherein the apertured film includes fluted holes.

5. A multiple layer water-absorbent core according claim 3 or 4 wherein the apertured film has fibers located on the upper surface.

6. A multiple layer water-absorbent core according to claim 1 or 2 wherein the water-management layer is a hydrophobic non-woven material.

7. A multiple layer water-absorbent core according claim 6 wherein the nonwoven material has fibers located on the upper surface.

8. A multiple layer water-absorbent core according to claim 5 or 7 wherein the fibers generally extend away from the water-management layer.

9. A multiple layer water-absorbent core according to any one of claim 5, 7 or 8 wherein voids exist between the fibers.

10. A multiple layer water-absorbent core according to any one of claim 5, 7, 8 or 9 wherein the fibers are arranged such that there are grooves or channels formed between the fibers or between groups of fibers.

11. A multiple layer water-absorbent core according to any one of claims 5, 7 to 10 wherein the fibers are selected from rayon fibers, cellulose ester fibers, protein fibers, polyamide fibers, polyester fibers, polyvinyl fibers, polyolefin fibers, polyurethane fibers, aramid fibers, glass fibers, fibers having a hollow core, bicomponent fibers and mixtures thereof.

12. An absorbent article comprising a liquid pervious cover sheet, a liquid impervious backsheet and a multiple layer water-absorbent core according to any one of claims 1 to 11 wherein the cover sheet is joined to the backsheet to enclose the water-absorbent core.

13. An absorbent article according to claim 12 wherein fibers are located on the upper surface of the water-management layer and the fibers are arranged such they extended in a direction substantially parallel to the longitudinal axis of the absorbent article.
